(19) United States
(12) Patent Application Publication
(10) Pub. No.: US 2026/0088183 A1
(43) Pub. Date: Mar. 26, 2026

(54) PREDICTION DEVICE, PREDICTION METHOD, AND NON-TRANSITORY COMPUTER- READABLE RECORDING MEDIUM

(71) Applicant: NEC Corporation, Tokyo (JP)

(72) Inventors: Yuki KOSAKA, Tokyo (JP); Kentaro NAKAHARA, Tokyo (JP); Keisuke SUZUKI, Tokyo (JP); Kosuke NISHIHARA, Tokyo (JP); Fumiyuki NIHEY, Tokyo (JP); Mana HASHIMOTO, Tokyo (JP)

(73) Assignee: NEC Corporation, Tokyo (JP)

(21) Appl. No.: 19/319,818

(22) Filed: Sep. 5, 2025

(30) Foreign Application Priority Data
Sep. 26, 2024 (JP) 2024-167201

(51) Int. Cl. G16H 50/30; G16H 10/60; G16H 50/70

(52) U.S. Cl. CPC G16H 50/30; G16H 10/60; G16H 50/70

(57) ABSTRACT

An aspect of the present disclosure includes: an acquisition unit acquiring health data being data regarding health of a plurality of persons in a first period and a second period a predetermined duration before the first period; a calculation unit calculating distribution feature information indicating a feature related to a distribution of health data of a first age group in the first period; a generation unit generating a prediction-target distribution by integrating a distribution of health data of a prediction-target age group corresponding to the age of the target person when the predetermined period of time or more has elapsed and the distribution feature information; and a prediction unit predicting the health data in the prediction-target distribution in the distribution of the health data of the first age group in the first period. The present disclosure supports decision making regarding the health of the target person.

This application is based upon and claims the benefit of priority from Japanese Patent Application No. 2024-167201, filed on Sep. 26, 2024, the disclosure of which is incorporated herein in its entirety by reference.

### TECHNICAL FIELD

The present disclosure relates to a prediction device and the like.

### BACKGROUND ART

In the field of healthcare and the like, there is a technology for performing future analysis of information regarding a person.

JP 2018-194904 A discloses a technology for predicting a state of a subject at a predetermined future time based on a medical examination record of the subject. Specifically, JP 2018-194904 A discloses that an output vector indicating prediction of a state of a subject at a predetermined future time is obtained by acquiring a past medical examination record of the subject, generating a plurality of input vectors based on the medical examination record, and inputting each of the input vectors to a machine learning algorithm.

### SUMMARY

According to an aspect of the present disclosure, there is provided a prediction device including an acquisition unit that acquires health data being data regarding health of a plurality of persons in a first period and a second period a predetermined duration before the first period, a calculation unit that calculates distribution feature information indicating a feature related to a distribution of health data of a first age group in the first period based on a relationship between the distribution of the health data of the first age group corresponding to an age of a target person, which is the distribution of the health data in the first period, and the distribution of the health data of the first age group or the distribution of the health data of an age group corresponding to the predetermined duration before the first age group, which is the distribution of the health data in the second period, a generation unit that generates a prediction-target distribution by integrating a distribution of health data of a prediction-target age group being the distribution in the first period and the distribution feature information, the prediction-target age group corresponding to the age of the target person in a case where the predetermined duration or more has elapsed, and a prediction unit that predicts the health data in the prediction-target distribution, which is a transition destination of data corresponding to the health data of the target person, in the distribution of the health data of the first age group in the first period.

According to another aspect of the present disclosure, there is provided a prediction method including acquiring health data being data regarding health of a plurality of persons in a first period and a second period a predetermined duration before the first period, calculating distribution feature information indicating a feature related to a distribution of health data of a first age group in the first period based on a relationship between the distribution of the health data of the first age group corresponding to an age of a target person, which is the distribution of the health data in the first period, and the distribution of the health data of the first age group or the distribution of the health data of an age group corresponding to the predetermined duration before the first age group, which is the distribution of the health data in the second period, generating a prediction-target distribution by integrating a distribution of health data of a prediction-target age group being the distribution in the first period and the distribution feature information, the prediction-target age group corresponding to the age of the target person in a case where the predetermined duration or more has elapsed, which is the distribution in the first period, and the distribution feature information, and predicting the health data in the prediction-target distribution, which is a transition destination of data corresponding to the health data of the target person, in the distribution of the health data of the first age group in the first period.

According to another aspect of the present disclosure, there is provided a non-transitory computer-readable recording medium storing a program that causes a computer to execute processing of acquiring health data being data regarding health of a plurality of persons in a first period and a second period a predetermined duration before the first period, processing of calculating distribution feature information indicating a feature related to a distribution of health data of a first age group in the first period based on a relationship between the distribution of the health data of the first age group corresponding to an age of a target person, which is the distribution of the health data in the first period, and the distribution of the health data of the first age group or the distribution of the health data of an age group corresponding to the predetermined duration before the first age group, which is the distribution of the health data in the second period, processing of generating a prediction-target distribution by integrating a distribution of health data of a prediction-target age group being the distribution in the first period and the distribution feature information, the prediction-target age group corresponding to the age of the target person in a case where the predetermined duration or more has elapsed, and processing of predicting the health data in the prediction-target distribution, which is a transition destination of data corresponding to the health data of the target person, in the distribution of the health data of the first age group in the first period.

### BRIEF DESCRIPTION OF THE DRAWINGS

FIG. 1 is a first block diagram illustrating an example of a functional configuration of a prediction device according to the present disclosure;

FIG. 2 is a first flowchart illustrating an example of an operation of a prediction device according to the present disclosure;

FIG. 3 is a second block diagram illustrating an example of a functional configuration of a prediction device according to the present disclosure;

FIG. 4 is a diagram illustrating an example of a probability density distribution according to the present disclosure;

FIG. 5 is a diagram illustrating an image in a case where a transition from one probability density distribution to another probability density distribution according to the present disclosure is solved as an optimal transport problem;

FIG. 6 is a second flowchart illustrating an example of an operation of a prediction device according to the present disclosure;

FIG. 7 is a third block diagram illustrating an example of a functional configuration of a prediction device according to the present disclosure;

FIG. 8 is a third flowchart illustrating an example of an operation of a prediction device according to the present disclosure;

FIG. 9 is a fourth flowchart illustrating an example of an operation of a prediction device according to the present disclosure; and

FIG. 10 is a block diagram illustrating an example of a hardware configuration of a computer device that implements a prediction device according to the present disclosure.

### EXAMPLE EMBODIMENT

Hereinafter, example embodiments of the present disclosure will be described with reference to the drawings.

### First Example Embodiment

An outline of a prediction device according to a first example embodiment will be described.

The prediction device of the present disclosure performs future prediction related to data based on a state transition between data elements, which is estimated using accumulated data. In the present disclosure, an example of target data is health data that is data regarding health of a person. That is, the prediction device in the present disclosure is possible to predict how the health data of the target person will transition in the future based on the state transition from the health data in one stage to the health data in another stage. Thus, the prediction device is possible to support decision making regarding the health of the target person.

The health data may include, for example, a value of an inspection item in a medical examination, or may include information regarding an exercise habit of a person. The health data is not limited to this example. For example, the health data is accumulated in advance. At this time, the accumulated health data may be data at a predetermined time point of each of a plurality of persons. For example, the results of the medical examinations for 10,000 people in a predetermined year may be accumulated as health data. In the present disclosure, an example in which the prediction device estimates the data transition based on the health data will be mainly described, but the target data is not limited to this example.

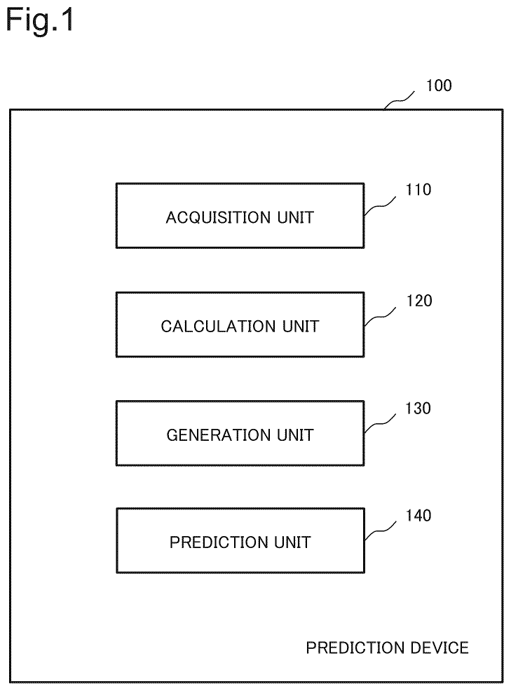

FIG. 1 is a first block diagram illustrating an example of a functional configuration of a prediction device 100. As illustrated in FIG. 1, the prediction device 100 includes an acquisition unit 110, a calculation unit 120, a generation unit 130, and a prediction unit 140.

The acquisition unit 110 acquires health data. More specifically, the acquisition unit 110 acquires health data in a first period. For example, the acquisition unit 110 acquires, as health data, results of medical examinations for a plurality of persons, which are performed in the year t. The acquisition unit 110 acquires health data in a second period. The second period is a period a predetermined duration before the first period. For example, the acquisition unit 110 acquires, as health data, results of medical examinations for a plurality of persons, which are performed in the year t−1.

The health data may be a dataset classified by stage. The stage may be information indicating a stratum resulting from stratifying the dataset. In other words, the stage can also be said to be information indicating a condition in a case where data serving as a population is classified into subsets based on a predetermined condition. For example, the dataset for each stage is health data for each age group. More specifically, in a case where the health data is data indicating blood glucose levels of a plurality of persons, the health data for each stage may include data indicating a blood glucose level of a person in the 10s age group, data indicating a blood glucose level of a person in 20s age group, . . . , and data indicating a blood glucose level of a person in 80s age group. That is, in this example, the dataset includes data indicating blood glucose levels for the 10s age group. In this manner, the order may be determined for the stage. For example, the next stage after the stage for the 10s age group is a stage for the 20s age group. The age group may be any age group. For example, the health data may be classified into data indicating blood glucose levels for each one-year age group.

The health data may be stored in a storage device (not illustrated). In this case, the storage device may be a device included in the prediction device 100 or an external device communicably connected to the prediction device 100.

The acquisition unit 110 acquires health data that is data regarding health of a plurality of persons in the first period and the second period a predetermined duration before the first period. The acquisition unit 110 is an example of an acquisition means.

The calculation unit 120 calculates distribution feature information. The distribution feature information indicates a feature related to a distribution of health data of a specific age group. For example, the distribution feature information indicates a feature related to a distribution of health data of the age group corresponding to the age of the target person. The age group corresponding to the age of the target person is referred to as a first age group.

The calculation unit 120 may calculate, as the distribution feature information, a difference between the distribution of the health data of the first age group in the first period and the distribution of the health data of the first age group in the second period. For example, the calculation unit 120 calculates a difference between the distribution of the health data of 50-year-old persons in the year t and the distribution of the health data of 50-year-old persons in the year t−1. An age group a predetermined duration before the first age group is defined as a second age group. At this time, the calculation unit 120 may calculate, as the distribution feature information, information based on transition from the distribution of the health data of the second age group in the second period to the distribution of the health data of the first age group in the first period. For example, the calculation unit 120 may use the transition from the distribution of the health data of 49-year-old persons in the year t−1 to the distribution of the health data of 50-year-old persons in the year t. That is, the calculation unit 120 calculates the distribution feature information based on a relationship between the distribution of the first age group in the first period and the distribution of the first age group in the second period or the distribution of the second age group in the second period. The example of calculating the distribution feature information is not limited to the above-described example.

The calculation unit 120 calculates the distribution feature information indicating a feature related to the distribution of the health data of the first age group in the first period based on the relationship between the distribution of the health data of the first age group corresponding to the age of the target person, which is the distribution of the health data in the first period, and the distribution of the health data of the first age group or the distribution of the health data of the age group a predetermined duration before the first age group, which is the distribution of the health data in the second period. The calculation unit 120 is an example of a calculation means.

The generation unit 130 generates a prediction-target distribution using the distribution feature information. It is assumed that it is desired to predict the health data of the target person in a case where a predetermined period of time or more has elapsed. The age group corresponding to the age of the target person when a predetermined period of time or more has elapsed is referred to as a prediction-target age group. The generation unit 130 generates the prediction-target distribution by integrating the distribution of the health data of the prediction-target age group in the first period and the distribution feature information. For example, it is assumed that the health data of a 50-year-old target person after 10 years is desired to be predicted. In this case, the generation unit 130 may integrate the distribution of the health data of 60-year-old persons in the first period and the distribution feature information. An example of the integration may be a linear combination of a matrix representing the distribution of the health data of the prediction-target age group and a matrix representing the distribution feature information. The method for generating the prediction-target distribution is not limited to this example.

The generation unit 130 generates the prediction-target distribution by integrating the distribution of the health data of the prediction-target age group corresponding to the age of the target person after a predetermined period of time, which is the distribution in the first period, and the distribution feature information. The generation unit 130 is an example of a generation means.

The prediction unit 140 predicts future health data of the target person using the prediction-target distribution. Specifically, the prediction unit 140 estimates the state transition from the distribution of the health data of the first age group in the first period to the prediction-target distribution.

It is assumed that the health data is data indicating blood glucose levels of a plurality of persons. It is assumed that the first age group is 50 years old. The prediction-target distribution is assumed to be a distribution generated by integrating the distribution of the health data of 60-year-old persons in the first period and the distribution feature information. In this case, the state transition of the data from the distribution of the blood glucose levels of the 50-year-old persons to the prediction-target distribution is estimated. At this time, the state transition may be estimated using an algorithm of the optimal transport problem. That is, a likely transition from a probability distribution indicating a probability that 50-year-old persons having values of the blood glucose levels are present to a probability distribution indicating a probability that 60-year-old persons having values of the blood glucose levels are present, may be estimated. In the estimation, a mapping from the probability distribution for 50-year-old persons to the probability distribution for 60-year-old persons is estimated. Estimating the mapping is synonymous with estimating a state transition probability related to a transition from a distribution of blood glucose levels of 50-year-old persons to a distribution of blood glucose levels of 60-year-old persons.

For example, using such a state transition probability, the prediction unit 140 calculates to which of the prediction-target distributions the data corresponding to the health data of the target person transitions in the distribution of the health data of the first age group. Thus, the prediction unit 140 may predict future health data of the target person.

The prediction unit 140 predicts the health data in the prediction-target distribution, which is the transition destination of the data corresponding to the health data of the target person, in the distribution of the health data of the first age group in the first period. The prediction unit 140 is an example of a prediction means.

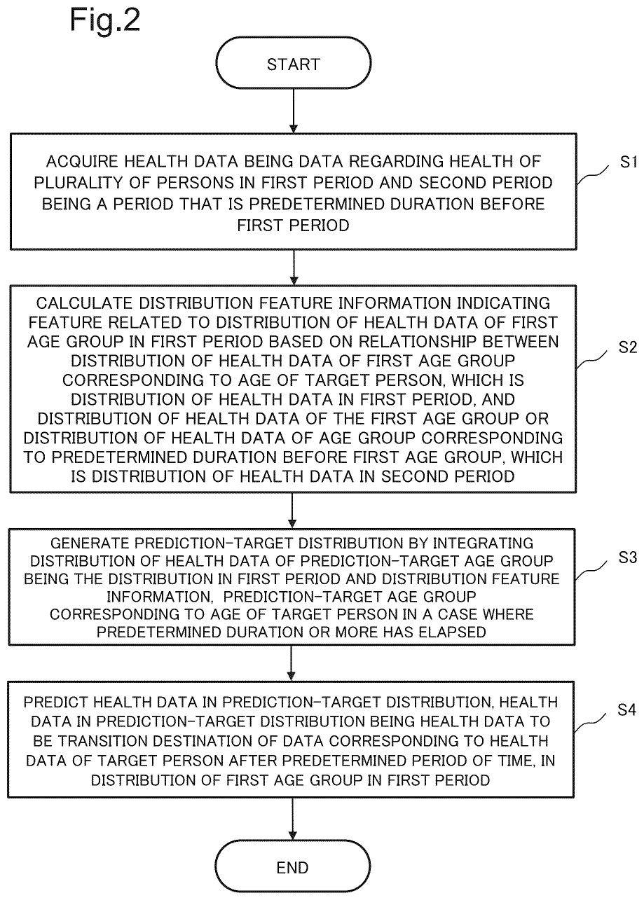

Next, an example of an operation of the prediction device 100 will be described with reference to FIG. 2. In the present disclosure, each step of the flowchart is represented using a number given to each step, such as “S1”.

FIG. 2 is a flowchart illustrating an example of the operation of the prediction device 100.

The acquisition unit 110 acquires health data being data regarding health of a plurality of persons in the first period and the second period, the second period being a period that is a predetermined duration before the first period (S1).

The calculation unit 120 calculates the distribution feature information indicating a feature related to the distribution of the health data of the first age group in the first period based on the relationship between the distribution of the health data of the first age group corresponding to the age of the target person, which is the distribution of the health data in the first period, and the distribution of the health data of the first age group or the distribution of the health data of the age group corresponding to a predetermined duration before the first age group, which is the distribution of the health data in the second period (S2).

The generation unit 130 generates the prediction-target distribution by integrating the distribution of the health data of the prediction-target age group being the distribution in the first period and the distribution feature information, the prediction-target age group corresponding to the age of the target person in a case where a predetermined duration or more has elapsed (S3).

The prediction unit 140 predicts the health data in the prediction-target distribution, the health data in the prediction-target distribution being the transition destination of the data corresponding to the health data of the target person, in the distribution of the health data of the first age group in the first period (S4).

The prediction device 100 of the first example embodiment acquires health data that is data regarding health of a plurality of persons in the first period and the second period a predetermined duration before the first period. The prediction device 100 calculates the distribution feature information indicating a feature related to the distribution of the health data of the first age group in the first period based on the relationship between the distribution of the health data of the first age group corresponding to the age of the target person, which is the distribution of the health data in the first period, and the distribution of the health data of the first age group or the distribution of the health data of the age group a predetermined duration before the first age group, which is the distribution of the health data in the second period. The prediction device 100 generates the prediction-target distribution by integrating the distribution of the health data of the prediction-target age group corresponding to the age of the target person when a predetermined period of time or more has elapsed, which is the distribution in the first period, and the distribution feature information. The prediction device 100 predicts the health data in the prediction-target distribution, which is the transition destination of the data corresponding to the health data of the target person, in the distribution of the health data of the first age group in the first period.

That is, the prediction device 100 performs prediction based on a transition from the distribution of the health data of the first age group in the first period to the distribution of the health data of the prediction-target age group. At this time, the prediction device 100 does not perform a method that requires temporal data obtained by observing the same person for a certain period of time in order to predict the data regarding the subject after the certain period of time. That is, even in a case where there is no temporal data for a predetermined period of time, the prediction device 100 can predict the health state after the lapse of a predetermined period of time or more.

The distribution feature information is added to the prediction-target distribution to be the transition destination. The distribution feature information is information indicating a feature related to the distribution of the health data of the first age group in the first period, which is a transition source. That is, the prediction device 100 can perform prediction in consideration of a distinctive feature regarding the distribution of the health data of the first age group in the first period is used.

### Second Example Embodiment

Next, a prediction device according to a second example embodiment will be described. In the second example embodiment, another example related to the prediction device described in the first example embodiment will be further described. Also in the second example embodiment, an example in which the prediction device performs prediction using the transition of the health data will be mainly described, but target data is not limited to an example to be described below. A part of the description of content overlapping with that of the first example embodiment will be omitted.

### [Details of Prediction Device 100]

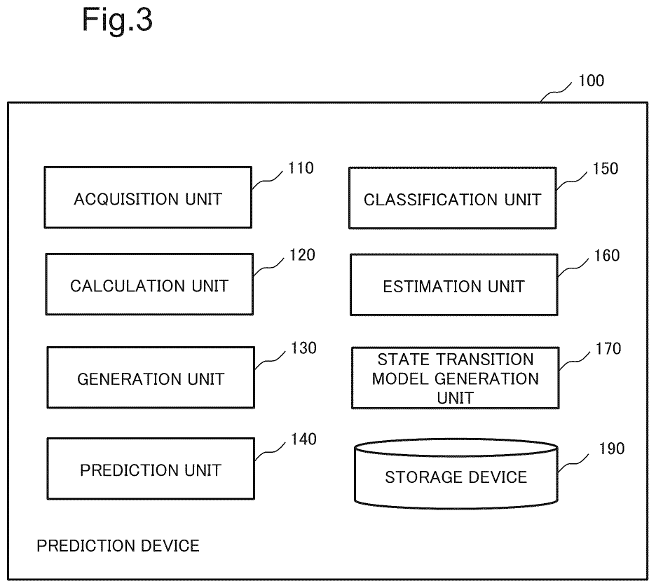

FIG. 3 is a block diagram illustrating an example of a functional configuration of a prediction device 100. The prediction device 100 includes an acquisition unit 110, a calculation unit 120, a generation unit 130, and a prediction unit 140. The prediction device 100 may include a classification unit 150 and an estimation unit 160. The prediction device 100 may include a state transition model generation unit 170. The prediction device 100 may include a storage device 190. The storage device 190 may be a device included in the prediction device 100 or an external device communicably connected to the prediction device 100.

The prediction device 100 is, for example, a device provided in a terminal device such as a personal computer. The terminal device is a device operated by a user. The prediction device 100 is not limited to this example, and may be a device implemented in a server device communicably connected to the terminal device via a wired or wireless network. The prediction device 100 may perform various types of processing in accordance with an instruction from the terminal device.

The prediction device 100 may be communicably connected to a further device via the wired or wireless network. For example, the prediction device 100 may be communicable with an external server device having health data. The external server device is, for example, a device managed by a hospital, a local government, a company, or the like.

The acquisition unit 110 acquires a dataset related to health data. At this time, the dataset is stored in the storage device 190. For example, the acquisition unit 110 may acquire the dataset related to the health data by reading the dataset stored in the storage device 190 in accordance with an instruction from the terminal device.

At this time, the data acquired in advance by the acquisition unit 110 may be stored in the storage device 190. Specifically, the acquisition unit 110 acquires the health data in the first period and the health data in the second period in advance from an external server device that manages the health data. For example, it is assumed that the results of the medical examinations for 10,000 persons in the year t and the year t−1 are managed by the external server device. The acquisition unit 110 acquires the results of the medical examinations for 10,000 persons in the year t and the year t−1 as health data from the external server device. The health data may be information corresponding to an inspection item of the medical examination. The health data may be a result of the medical examination that each of the 10,000 persons undergoes at one time point in one year. For example, the health data in the year t may include the results of the medical examinations that 10,000 persons undergo at one time point in the year t. For example, the health data in the year t−1 may include the results of the medical examinations that 10,000 persons undergo at one time point in the year t−1. As described above, the dataset during a certain period of time may be data measured at a time point for each of a plurality of subjects instead of data indicating a temporal change of the same subject. The person in the health data in the year t may be different from the person in the health data in the year t−1. The number of persons in the health data in the year t may be different from the number of persons in the health data in the year t−1. The acquisition unit 110 stores the acquired health data in the storage device 190.

The predetermined period of time, which is the interval between the first period and the second period, may not be one year. The predetermined period of time may be two years or more or less than one year. The method for acquiring the health data is not limited to this example. For example, there may be a recording medium storing health data. At this time, the terminal device reads the health data from the recording medium. The acquisition unit 110 may acquire the health data read by the terminal device.

For example, the health data is processed by the classification unit 150. The processed health data may be stored in the storage device 190. The classification unit 150 processes the data acquired by the acquisition unit 110 into a dataset according to the condition. For example, the classification unit 150 classifies the health data for each age group. For example, the classification unit 150 classifies the health data for each one-year age group. The present disclosure is not limited to this example, and the classification unit 150 may classify the health data at an arbitrary age interval. For example, the classification unit 150 may classify the health data in 10-year age intervals.

At this time, the classification unit 150 may extract specific data from the health data and classify the extracted data for each age group. For example, it is assumed that the health data includes information indicating height, weight, blood pressure, blood glucose level, HbA1c, and Body Mass Index (BMI). At this time, the classification unit 150 may classify data indicating the blood glucose level and BMI among pieces of the health data for each age group.

The condition for classification and the data to be extracted may be information in accordance with an instruction from the terminal device. That is, a user who operates the terminal device inputs information indicating a condition for classification and data to be extracted to the terminal device. The terminal device transmits the input information to the prediction device 100. The classification unit 150 processes the health data using the information indicating the condition for classification and the data to be extracted transmitted from the terminal device.

The classification unit 150 generates a distribution related to the acquired data. Specifically, the classification unit 150 generates a probability density distribution for each condition based on the acquired data. For example, the classification unit 150 generates a distribution obtained by plotting data indicating the blood glucose level and BMI for each age group. The distribution generated at this time is a two-dimensional distribution related to the blood glucose level and BMI. Then, the classification unit 150 generates a probability density distribution indicating an existence probability of respective values of blood glucose level and BMI When a data value of one dimension is xᵢ and a data value of another dimension is xⱼ, the probability density distribution can be represented as p([xᵢ, xⱼ]). The classification unit 150 generates a probability density distribution for each age group related to the acquired health data.

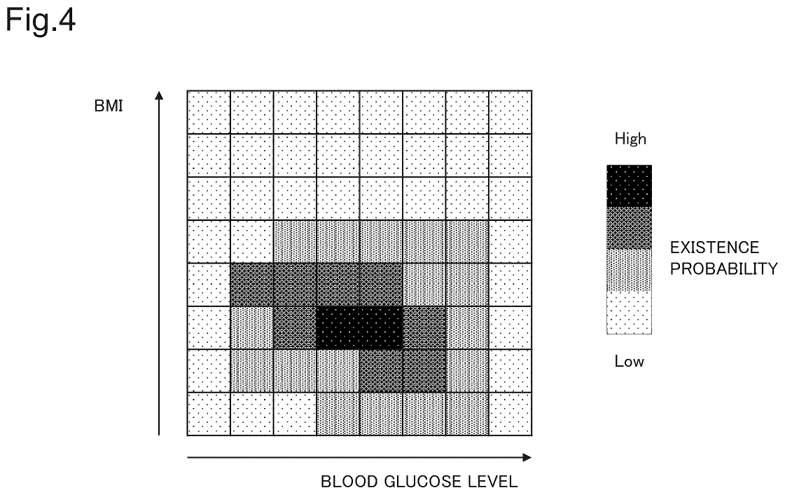

At this time, the classification unit 150 may classify data in each distribution into data groups. In this case, the probability density distribution is a distribution indicating the existence probability for each data group. FIG. 4 is a diagram illustrating an example of the probability density distribution. The probability density distribution illustrated in FIG. 4 has the blood glucose level and BMI as respective axes. In the example of FIG. 4, 64 cells are illustrated. This cell is a data group obtained by classifying data indicating the blood glucose level and BMI of each person. Then, the existence probability for each data group is illustrated. The classification unit 150 may classify data in each distribution of health data for each age group in each period into data groups. The classification unit 150 is an example of a classification means.

The classification unit 150 may generate a distribution of two or more dimensions. For example, the classification unit 150 may generate a probability density distribution having blood glucose level, BMI, and average daily step count as respective axes. The probability density distribution divided by the cells as illustrated in FIG. 4 is relevant to a marginal distribution based on each value of the health data. The probability density distribution to be treated hereinafter may be a probability distribution as illustrated in FIG. 4 or a probability distribution that does not take the form of the marginal distribution.

The classification unit 150 stores again the health data processed as described above in the storage device 190. For example, the classification unit 150 may store the probability density distribution related to the health data for each age group in each period in the storage device 190.

The acquisition unit 110 acquires the health data classified for each condition as a dataset for each stage. For example, the acquisition unit 110 may acquire the probability density distribution related to the health data for each age group as described above as a dataset for each stage. That is, the acquisition unit 110 may acquire the probability density distribution related to the health data for each age group in the first period and the probability density distribution related to the health data for each age group in the second period.

The acquisition unit 110 acquires health data of the target person. The health data of the target person is also referred to as target data. For example, in a case where the probability density distribution related to the blood glucose level and BMI is acquired, the acquisition unit 110 acquires target data indicating the blood glucose level and BMI of the target person. At this time, the target data includes information indicating the age of the target person.

The acquisition unit 110 may acquire further information. For example, the acquisition unit 110 may acquire health data in another period.

The calculation unit 120 calculates the distribution feature information indicating a feature related to the distribution of the health data of the first age group in the first period. The generation unit 130 generates a prediction-target distribution using the calculated distribution feature information. At this time, the prediction-target distribution may be a probability density distribution obtained by adding the distribution feature information to health data in the prediction-target age group. Details of the method for calculating the distribution feature information and the method for generating the prediction-target distribution will be described later.

The prediction unit 140 predicts the transition of the health data of the target person. In other words, the prediction unit 140 predicts the value of the future health data of the target person based on the target data. Specifically, the prediction unit 140 predicts the value of the health data in a case where the target person has reached the age corresponding to the prediction-target age group. At this time, the first age group is an age group corresponding to the age of the target person.

For example, it is assumed that the target person is 51 years old. In this case, the age of the target person corresponds to the first age group. The prediction unit 140 specifies which data or a data group the target data is classified into in the probability density distribution related to the health data of 51-year-old persons in the first period. The prediction unit 140 predicts to which data or a data group the specified data or data group transitions in the prediction-target distribution based on the state transition probability.

The state transition probability is estimated by the estimation unit 160. The estimation unit 160 estimates the state transition probability between the distributions. Specifically, the estimation unit 160 estimates the state transition probability based on the transition from the distribution of the health data of the first age group in the first period to the prediction-target distribution. The prediction-target age group corresponds to an age group after a predetermined period of time or more has elapsed since the first age group. For example, it is assumed that the first period is the year t. It is assumed that the second period, which is a period a predetermined duration before the first period, is the year t−1. That is, it is assumed that a difference between the first period and the second period is one year. At this time, in a case where the first age group corresponds to age 51, the prediction-target age group corresponds to age 52 or older. Hereinafter, the distribution of the health data of the first age group in the first period is also simply referred to as a first distribution.

The estimation unit 160 estimates the transition from the first distribution to the prediction-target distribution using an algorithm of an optimal transport problem (hereinafter, referred to as an optimal transport algorithm). The optimal transport algorithm is an algorithm for obtaining a transport method for optimizing a cost necessary for transitioning a predetermined probability distribution to another probability distribution.

Specifically, the fact that for distributions u and v on a probability space X, a distribution π on a direct product X² is a coupling indicates that Expressions 1 and 2 below hold.

$$π ( \cdot \times X ) = μ \qquad [ Expression 1 ]$$

$$π ( X \times \cdot ) = v \qquad [ Expression 2 ]$$

The entire coupling is defined as Π(μ, v). A cost function for transporting an element x included in the distribution μ to an element y included in the distribution vis denoted as c(x, y). In this case, for example, in Expression 3 below, the coupling that minimizes the cost is referred to as optimal transport.

$$\begin{matrix}{\int }_{{X}^{2}} c ( x , y ) π ( dxdy ) , & π ∈ \prod ( μ , v )\end{matrix} \qquad [ Expression 3 ]$$

Assuming that the mapping from the distribution u to the distribution v is T(x), the direct transition may be obtained by finding T that minimizes Expression 4 below. In this case, T is a one-to-one mapping. It is assumed that for a subset U of u, the volume of a mapping T(U) is equal to that of U.

$${\int }_{{X}^{2}} c ( x , T ( x ) ) d μ ( x ) \qquad [ Expression 4 ]$$

In a case where optimal transport is performed for discrete data, it can also be formulated as follows. Specifically, Cᵢⱼ is a cost matrix, and distributions are μᵢ and vⱼ. At this time, Pᵢⱼ that minimizes Expression 5 indicating the total cost is obtained under the condition shown in Expression 6.

$${\sum }_{i = 1}^{n} {\sum }_{j = 1}^{n} {C}_{ij} {P}_{ij} \qquad [ Expression 5 ]$$

$${\sum }_{j = 1}^{n} {P}_{ij} = {μ}_{i} , {\sum }_{i = 1}^{n} {P}_{ij} = {v}_{j} , {P}_{ij} \geq 0 , ∀ i , j \qquad [ Expression 6 ]$$

It is possible to calculate a set of pre-transport data and destination data, which optimizes the transport cost from the first distribution to the prediction-target distribution by the optimal transport algorithm.

The estimation unit 160 estimates the state transition probability based on the transition from the first distribution (that is, the distribution of the health data of the first age group in the first period) to the prediction-target distribution. At this time, the estimation unit 160 solves the transition from the first distribution to the prediction-target distribution as the optimal transport problem.

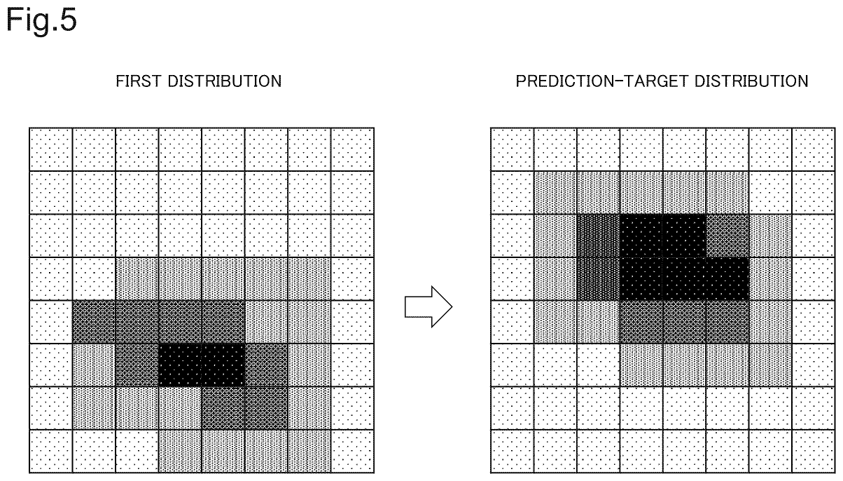

FIG. 5 is a diagram illustrating an image in a case where the transition from one probability density distribution to another probability density distribution is solved as an optimal transport problem. FIG. 5 illustrates the probability density distribution related to the health data of the first age group in the first period and the prediction-target The prediction-target distribution can also be referred to as a probability density distribution of the prediction-target age group. Solving, by the estimation unit 160, the transition from the first distribution to the prediction-target distribution as the optimal transport problem is relevant to estimating, for each cell in the probability density distribution of the first age group, which cell in the probability density distribution of the prediction-target age group it is most likely to transition to. That is, the estimation unit 160 estimates the state transition probability based on the transition from each data group in the first distribution to each data group in the prediction-target distribution.

For example, μ is a probability density distribution of the first age group, and vis a probability density distribution (prediction-target distribution) of the prediction-target age group. At this time, the estimation unit 160 estimates a mapping T using, for example, Expression 4. For example, the estimation unit 160 models the mapping T as a function using a neural network such as a fully-connected multilayer. The estimation unit 160 obtains the mapping T by performing optimization using machine learning in such a way that Expression 3 becomes small. The estimation unit 160 generates a plurality of y's transitioning from the given x using the mapping T. The estimation unit 160 obtains the state transition probability from the generated y's. Thus, the estimation unit 160 estimates the state transition probability. The function of the estimation unit 160 may be included in the prediction unit 140.

The prediction unit 140 predicts the data group of the health data in the prediction-target distribution, which is the data group to be the transition destination of the data group obtained by classifying the health data of the target person, in the distribution of the health data of the first age group in the first period. For example, the prediction unit 140 predicts the transition of the health data of the target person using the state transition probability estimated in this manner.

Next, a specific example regarding generation of the prediction-target distribution will be described. In the following example, the first period is set to the year t, and the second period a predetermined duration before the first period is set to the year t−1. The first age group corresponds to age 51, and the second age group corresponds to age 50. The second age group is an age group a predetermined duration before the first age group. The prediction-target age group corresponds to age 70. That is, in the following example, an example of generating a prediction-target distribution in a case of estimating the transition from the distribution of the health data of 51-year-old persons to the distribution of the health data of 70-year-old persons in the year t will be described. It is assumed that the storage device 190 stores the probability density distribution of the health data for each age in the year t−1 and the probability density distribution of the health data for each age in the year t. Hereinafter, the distribution of the health data is also simply referred to as “distribution”.

### [First Example Regarding Generation of Prediction-Target Distribution]

In a first example, an example in which a prediction-target distribution is generated using distribution feature information based on a relationship between the first distribution (the distribution of the first age group in the first period) and the distribution of the first age group in the second period will be described. Specifically, the prediction device 100 generates the prediction-target distribution by applying the state transition model generated based on the distribution of the first age group in the second period to the first distribution.

In this example, the prediction device 100 may include a state transition model generation unit 170. The state transition model generation unit 170 generates a state transition model for predicting a transition from a distribution of health data of one age group in the second period to a distribution of health data of another age group in the first period. The another age group is an age group a predetermined duration after the one age group.

For example, the state transition model generation unit 170 specifies the distribution of the health data of 51-year-old persons in the year t−1. The state transition model generation unit 170 specifies the distribution of the health data of 52-year-old persons in the year t. The state transition model generation unit 170 generates a state transition model indicating a state transition probability of the health data in a case where the 51-year-old person turned 52 based on the distribution of the health data of the 51-year-old persons in the year t−1 and the distribution of the health data of the 52-year-old persons in the year t. The state transition model at this time is represented as P(X₅₂|X₅₁). In this example, the predetermined period of time is one year. Therefore, the state transition model in a case where a person in one age group i (i is a natural number) transitions to another age group i+1 can be represented by Expression 7 below.

$$P ( {X}_{i + 1} \vert {X}_{i} ) \qquad [ Expression 7 ]$$

The state transition model generation unit 170 generates a state transition model corresponding to each age group. At this time, the state transition model generation unit 170 may generate the state transition model in a range of the age group where i is an age group a predetermined duration before the prediction-target age group relative to the first age group. In this example, the state transition model generation unit 170 may generate the state transition model in a range of (51≤i<70).

As described above, the state transition model generation unit 170 generates the state transition model for predicting the transition of the distribution in a case of transition from one age group to another age group based on the relationship between the distribution of the health data of one age group in the second period and the distribution of the health data of another age group a predetermined duration after the one age group in the first period. The state transition model generation unit 170 is an example of a state transition model generation means. The calculation unit 120 may have the function of the state transition model generation unit 170.

The calculation unit 120 calculates the distribution feature information using the generated state transition model. For example, the calculation unit 120 applies P(X₅₂|X₅₁) to the first distribution. Thus, the distribution for 52-year-old persons based on the first distribution is predicted. The calculation unit 120 applies P(X₅₃|X₅₂) to the predicted distribution for 52-year-old persons. Thus, the distribution for 53-year-old persons based on the predicted distribution for the 52-year-old persons is predicted. The calculation unit 120 performs similar processing until the distribution for 70-year-old persons corresponding to the prediction-target age group is predicted. The calculation unit 120 calculates the predicted distribution for 70-year-old persons as distribution feature information.

The calculation unit 120 calculates the distribution of the prediction-target age group estimated from the first distribution using the state transition model for predicting the transition of the distribution in a case of the age group a predetermined duration after the one age group. The distribution of the prediction-target age group at this time corresponds to a post-transition distribution indicating the transition destination of the first distribution when using the state transition model based on the accumulated health data over two periods.

That is, the calculation unit 120 calculates, as the distribution feature information, the post-transition distribution indicating the transition destination of the distribution of the health data of the first age group in the first period in a case of the transition from the first age group to the prediction-target age group, using the state transition model.

The generation unit 130 integrates the calculated distribution feature information and the distribution of the health data of the prediction-target age group in the first period. For example, each distribution is represented by a matrix M. At this time, the distribution of the health data of the prediction-target age group in the first period is denoted as Mb. The distribution feature information in this example is denoted as M_{f}. The prediction-target distribution is denoted as Mₚ. In this case, the prediction-target distribution Mₚ can be represented by Expression 8 below.

$$\begin{matrix}{M}_{p} = α {M}_{b} + ( 1 - α ) {M}_{f} & ( 0 \leq α \leq 1 )\end{matrix} \qquad [ Expression 8 ]$$

α is a coefficient. The generation unit 130 may generate a prediction-target distribution as the linear combination of the matrices. The present disclosure is not limited to this example, and each distribution may be expressed as a list. α may be arbitrarily determined. For example, it may be set based on the prediction-target distribution generated in the past and the true value of the distribution of the prediction-target age group. Specifically, it is assumed that a prediction-target distribution for 70-year-old persons is generated in the past using the distribution for 51-year-old persons in the year t′. It is assumed that the prediction device 100 has health data in the year t′+19. At this time, the prediction device 100 adjusts the coefficient α in such a way that Expression for generating the prediction-target distribution in the past calculates a distribution similar to the distribution of the prediction-target age group in the year t′+19. The prediction device 100 may use the adjusted a when generating the prediction-target distribution Mₚ.

### [Second Example Regarding Generation of Prediction-Target Distribution]

In a second example, another example in which a prediction-target distribution is generated using distribution feature information based on a relationship between the first distribution (the distribution of the first age group in the first period) and the distribution of the first age group in the second period will be described. Specifically, the prediction device 100 generates the prediction-target distribution using a difference between the first distribution and the distribution of the first age group in the second period.

For example, the calculation unit 120 specifies a distribution for 51-year-old persons in the year t and a distribution for 51-year-old persons in the year t−1. The calculation unit 120 calculates a difference between the distribution for 51-year-old persons in the year t and the distribution for 51-year-old persons in the year t−1. The difference is referred to as a first difference.

The distribution for 51-year-old persons in the year t is represented by a matrix M_{t_51}. The distribution for 51-year-old persons in the year t−1 is represented by a matrix M_{t-1_51}. The first difference is denoted as D₁. At this time, the first difference is calculated using Expression 9 below.

$${D}_{1} = {M}_{t\_ 51} - {M}_{t - 1 \_ 51} \qquad [ Expression 9 ]$$

The calculation unit 120 may calculate the first difference as the distribution feature information. That is, the calculation unit 120 may calculate, as the distribution feature information, the first difference which is a difference between the distribution of the health data of the first age group in the first period and the distribution of the health data of the first age group in the second period.

The generation unit 130 integrates the calculated first difference and the distribution of the health data of the prediction-target age group in the first period. For example, the prediction-target distribution Mₚ can be represented by Expression 10 below.

$$\begin{matrix}{M}_{p} = α {M}_{b} + ( 1 - α ) {D}_{1} & ( 0 \leq α \leq 1 )\end{matrix} \qquad [ Expression 10 ]$$

Similarly to the first example, the generation unit 130 may generate a prediction-target distribution as the linear combination of the matrices. The present disclosure is not limited to this example, and each distribution may be expressed as a list. α may be arbitrarily determined.

The method for generating the prediction-target distribution using the difference is not limited to this example. Specifically, the calculation unit 120 may calculate the distribution feature information by combining the first difference and the second difference.

The second difference is a difference between the distribution of the first age group in the second period and the distribution of the previous prediction-target age group in the second period. For example, the calculation unit 120 specifies a distribution for 51-year-old persons in the year t−1 and a distribution for 70-year-old persons in the year t−1. The calculation unit 120 calculates a difference between the distribution for 51-year-old persons in the year t−1 and the distribution for 70-year-old persons in the year t−1.

The distribution for 51-year-old persons in the year t−1 is represented by a matrix M_{t-1_51}. The distribution for 70-year-old persons in the year t−1 is represented by a matrix M_{t-1_70}. The first difference is denoted as D₂. At this time, the second difference is calculated using Expression 11 below.

$${D}_{2} = {M}_{t - 1 \_ 70} - {M}_{t - 1 \_ 51} \qquad [ Expression 11 ]$$

The calculation unit 120 calculates, as the distribution feature information, information obtained by combining the first difference and the second difference. The generation unit 130 integrates the calculated distribution feature information and the distribution of the health data of the prediction-target age group in the first period. For example, the prediction-target distribution Mₚ can be represented by Expression 12 below.

$$\begin{matrix}{M}_{p} = α {M}_{b} + ( 1 - α ) ( {D}_{1} + {D}_{2} ) & ( 0 \leq α \leq 1 )\end{matrix} \qquad [ Expression 12 ]$$

### [Third Example Regarding Generation of Prediction-Target Distribution]

In a third example, an example in which a prediction-target distribution is generated using distribution feature information based on a relationship between the first distribution (the distribution of the first age group in the first period) and the distribution of the health data of the age group a predetermined duration before the first age group in the second period will be described. Specifically, the prediction device 100 generates the prediction-target distribution using a difference between the first distribution and the prediction distribution of the first age group predicted based on the distribution of the second age group in the second period.

In this example, the acquisition unit 110 may acquire in advance the prediction distribution of the first age group predicted based on the distribution of the second age group in the second period. For example, the acquisition unit 110 acquires, as the prediction distribution, a distribution for 51-year-old persons predicted based on the distribution for 50-year-old persons in the year t−1. The prediction distribution may be a distribution generated by an arbitrary method. For example, the prediction distribution may be a distribution generated using a machine learning model generated based on the past health data. As an example, the prediction distribution may be a distribution for 51-year-old persons predicted from a distribution for 50-year-old persons in the year t−1 using a linear model. The method for generating the prediction distribution is not limited to this example. The prediction distribution is only required to be a distribution generated by the machine learning model based on the health data in the second period or before the second period, or the machine learning model based on the health data of another dataset.

The calculation unit 120 calculates, as the distribution feature information, a difference between the prediction distribution of the first age group predicted based on the distribution of the second age group in the second period and the first distribution. It is assumed that the distribution for 51-year-old persons predicted based on the distribution for 50-year-old persons in the year t−1 is acquired as the prediction distribution. The prediction distribution corresponds to the distribution for 51-year-old persons in the year t predicted from the health data in the year t−1. On the other hand, the distribution for 51-year-old persons in the year t can be regarded as a true value for the prediction distribution. That is, the difference between the prediction distribution and the distribution for 51-year-old persons in the year t corresponds to a prediction error.

The distribution for 51-year-old persons in the year t is represented by a matrix M_{t_51}. The distribution for 51-year-old persons predicted from the health data of the 50-year-old persons in the year t−1 is represented by a matrix M_{p_51}. The prediction error is denoted as D₃. At this time, the prediction error is calculated using Expression 13 below.

$${D}_{3} = {M}_{t \_ 51} - {M}_{p \_ 51} \qquad [ Expression 13 ]$$

The calculation unit 120 may calculate, as the distribution feature information, a difference between the prediction distribution of the first age group predicted based on the distribution of the age group a predetermined duration before the first age group in the second period and the distribution of the first age group in the first period.

The generation unit 130 integrates the calculated prediction error and the distribution of the health data of the prediction-target age group in the first period. For example, the prediction-target distribution Mₚ can be represented by Expression 14 below.

$$\begin{matrix}{M}_{p} = α {M}_{b} + ( 1 - α ) {D}_{3} & ( 0 \leq α \leq 1 )\end{matrix} \qquad [ Expression 14 ]$$

Similarly to the first example, the generation unit 130 may generate a prediction-target distribution as the linear combination of the matrices. The present disclosure is not limited to this example, and each distribution may be expressed as a list. α may be arbitrarily determined.

### [Operation Example of Prediction Device 100]

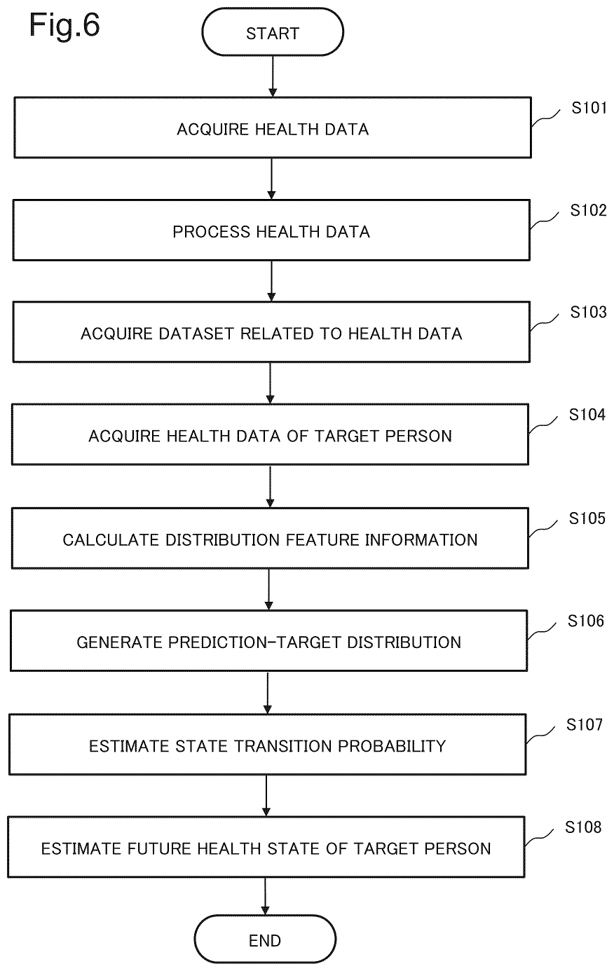

Next, an example of an operation of the prediction device 100 will be described with reference to FIG. 6.

FIG. 6 is a second flowchart illustrating an example of the operation of the prediction device 100. Specifically, FIG. 6 is a flowchart illustrating an example of the operation when the prediction device 100 predicts the health data of the future target person using the generated prediction-target distribution.

The acquisition unit 110 acquires health data (S101). For example, the acquisition unit 110 acquires the health data in the first period and the second period from an external server device. The acquisition unit 110 stores the health data in the storage device 190. The classification unit 150 processes the health data (S102). For example, the classification unit 150 classifies the health data in each period for each age group.

The classification unit 150 generates a probability density distribution for each one-year age group regarding the health data.

The acquisition unit 110 acquires a dataset related to the health data (S103). Specifically, the acquisition unit 110 acquires, from the storage device 190, probability density distributions for each one-year age group regarding the health data in the first period and the second period. The acquisition unit 110 acquires target data that is the health data of a target person (S104). The target data may be stored in advance in the storage device 190. The acquisition unit 110 may acquire the target data from an external server or from a terminal device.

The calculation unit 120 calculates the distribution feature information (S105). For example, the calculation unit 120 generates the distribution feature information by applying the state transition model generated based on the distribution of the first age group in the second period to the first distribution. At this time, the state transition model may be generated by the state transition model generation unit 170. The calculation unit 120 may calculate, as the distribution feature information, a difference between the first distribution and the distribution of the first age group in the second period. The calculation unit 120 may calculate, as the distribution feature information, a difference between the prediction distribution of the first age group predicted based on the distribution of the second age group in the second period and the first distribution.

The generation unit 130 generates a prediction-target distribution (S106). For example, the generation unit 130 generates the prediction-target distribution by integrating the calculated distribution feature information and the distribution of the health data of the prediction-target age group in the first period.

The estimation unit 160 estimates the state transition probability (S107). Specifically, the estimation unit 160 estimates the state transition probability based on the transition from the distribution (first distribution) of the health data of the first age group in the first period to the prediction-target distribution. At this time, the state transition probability may be estimated using an optimal transport algorithm.

The prediction unit 140 predicts the health state of the target person (S108). For example, the prediction unit 140 specifies data or a data group in the first distribution relevant to the target data. The prediction unit 140 predicts the health data in a case where the target person reaches the prediction-target age group based on the state transition probability. For example, the prediction unit 140 predicts a data group in the prediction-target distribution, which is a transition destination of the specified data group based on the state transition probability, as the health data in a case where the target person reaches the age corresponding to the prediction-target age group.

The present operation example is merely an example. That is, the operation of the prediction device 100 of the present disclosure is not limited to this example.

The prediction device 100 of the second example embodiment acquires the health data that is data regarding health of a plurality of persons in the first period and the second period a predetermined duration before the first period. The prediction device 100 calculates the distribution feature information indicating a feature related to the distribution of the health data of the first age group in the first period based on the relationship between the distribution of the health data of the first age group corresponding to the age of the target person, which is the distribution of the health data in the first period, and the distribution of the health data of the first age group or the distribution of the health data of the age group a predetermined duration before the first age group, which is the distribution of the health data in the second period. The prediction device 100 generates the prediction-target distribution by integrating the distribution of the health data of the prediction-target age group corresponding to the age of the target person when a predetermined period of time or more has elapsed, which is the distribution in the first period, and the distribution feature information. The prediction device 100 predicts the health data in the prediction-target distribution, which is the transition destination of the data corresponding to the health data of the target person, in the distribution of the health data of the first age group in the first period.

That is, the prediction device 100 performs prediction based on a transition from the distribution of the health data of the first age group in the first period to the distribution of the health data of the prediction-target age group. At this time, the prediction device 100 does not perform a method that requires temporal data obtained by observing the same person for a certain period of time in order to predict the data regarding the subject after the certain period of time. That is, even in a case where there is no temporal data for a predetermined period of time, the prediction device 100 can predict the health state after the lapse of a predetermined period of time or more.

The distribution feature information is added to the prediction-target distribution to be the transition destination. The distribution feature information is information indicating a feature related to the distribution of the health data of the first age group in the first period, which is a transition source. That is, the prediction device 100 can perform prediction in consideration of a distinctive feature regarding the distribution of the health data of the first age group in the first period is used.

For example, the prediction device 100 generates the state transition model for predicting the transition of the distribution in a case of transition from one age group to another age group based on the relationship between the distribution of the health data of one age group in the second period and the distribution of the health data of another age group a predetermined duration after the one age group in the first period. The prediction device 100 calculates, as the distribution feature information, the post-transition distribution indicating the transition destination of the distribution of the health data of the first age group in the first period in a case of the transition from the first age group to the prediction-target age group, using the state transition model.

For example, the prediction device 100 calculates, as the distribution feature information, the first difference which is a difference between the distribution of the health data of the first age group in the first period and the distribution of the health data of the first age group in the second period.

The prediction device 100 may calculate the distribution feature information by combining the first difference and the second difference. At this time, the second difference is a difference between the distribution of the first age group in the second period and the distribution of the prediction-target age group in the second period.

For example, the prediction device 100 calculates, as the distribution feature information, a difference between the prediction distribution of the first age group predicted based on the distribution of the age group a predetermined duration before the first age group in the second period and the distribution of the first age group in the first period.

The prediction-target distribution to which such distribution feature information is added is a distribution obtained by correcting the distribution of the health data of the prediction-target age group in the first period. That is, the distribution feature information corresponds to the role of the correction information. Therefore, the prediction device 100 can perform prediction with higher accuracy than that in a case where the transition destination of the health data of the first age group in the first period is simply the health data of the prediction-target age group in the first period.

### Third Example Embodiment

Next, a prediction device according to a third example embodiment will be described. Also in the third example embodiment, an example in which the prediction device performs prediction using the transition of the health data will be mainly described, but target data is not limited to an example to be described below. The description of part of the content overlapping with the content of the first example embodiment and the second example embodiment will be omitted.

### [Details of Prediction Device 101]

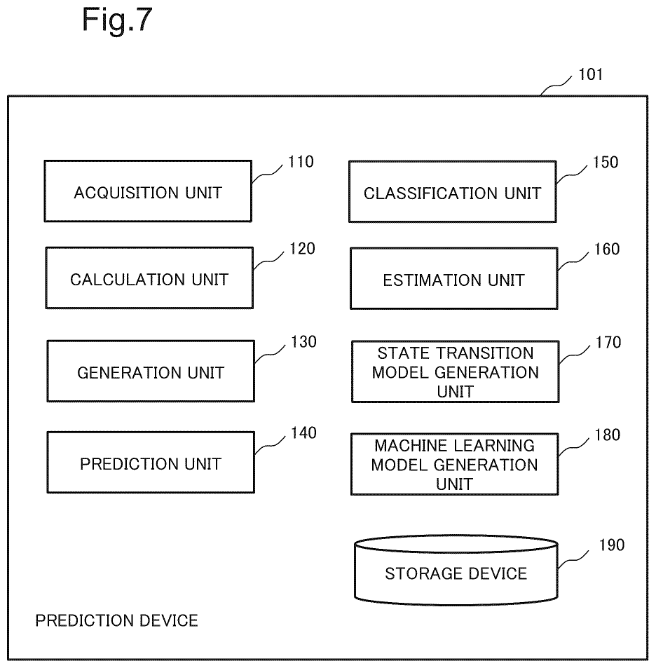

A prediction device 101 is a device in which additional functional units are added to the prediction device 100. FIG. 7 is a block diagram illustrating an example of a functional configuration of the prediction device 101. The prediction device 101 includes an acquisition unit 110, a calculation unit 120, a generation unit 130, a prediction unit 140, a classification unit 150, and an estimation unit 160. The prediction device 101 may include a state transition model generation unit 170. The prediction device 101 may include a machine learning model generation unit 180. The prediction device 101 may include a storage device 190.

Similarly to the prediction device 100, the prediction device 101 may be a device provided in a terminal device, or may be a device implemented in a server device communicably connected to the terminal device via a wired or wireless network.

The prediction device 101 calculates a state transition probability between stages in advance. The prediction device 101 generates a machine learning model using the calculated state transition probability. The prediction device 101 predicts future health data for the target person using the generated machine learning model. In the present example embodiment, a stage of generating the machine learning model is referred to as a generation phase. A stage of performing prediction is referred to as a prediction phase.

### (Generation Phase)

It is assumed that probability density distributions related to health data for each age group in the first period and second period are stored in the storage device 190.

That is, the acquisition unit 110 acquires the probability density distribution related to the health data for each age group in the first period and the probability density distribution related to the health data for each age group in the second period.

The calculation unit 120 calculates the distribution feature information corresponding to each age group. Specifically, the transition from the distribution of one age group in the first period to the distribution of each age group above the one age group is considered. That is, the calculation unit 120 calculates the distribution feature information corresponding to a case where each age group above the one age group is set as the prediction-target age group. For example, the distribution of the transition source is a distribution for 50-year-old persons. At this time, the calculation unit 120 calculates the distribution feature information corresponding to a case of considering the transition from the distribution for 50-year-old persons to the distribution of each age group of age 51 or older.

The calculation unit 120 similarly calculates the distribution feature information even in a case where the age group different from age 50 is set as the distribution of the transition source. In other words, the calculation unit 120 calculates the distribution feature information for each combination of the age group of the transition source and the prediction-target age group in the acquired health data. The age group of the transition source corresponds to the role of the first age group.

The generation unit 130 generates the prediction-target distribution for each combination of the age group of the transition source and the prediction-target age group.

The estimation unit 160 estimates the state transition probability for each combination of the age group of the transition source and the prediction-target age group based on the generated prediction-target distribution. More specifically, the estimation unit 160 estimates the state transition probability based on the transition of the distribution of the health data for each combination of the age group of the transition source and the prediction-target age group in the health data in the first period.

The machine learning model generation unit 180 generates a machine learning model. Specifically, the machine learning model generation unit 180 generates a prediction model that outputs data in another stage, which is a transition destination of data in one stage, based on the estimated state transition probability. The prediction model corresponds to a machine learning model that is trained on a relationship between a data distribution in one stage and a data distribution in another stage. The machine learning model generation unit 180 may generate a prediction model that outputs a data group of data in another stage, which is a transition destination of a data group of a data distribution in one stage, based on the estimated state transition probability. The generated prediction model is a machine learning model that receives age and health data as inputs, and outputs health data to which the input health data transitions after a predetermined period of time or a data group thereof.

The machine learning model generation unit 180 generates the machine learning model trained on the relationship between the data in the stage of the transition source and the data in the stage after transition based on the state transition probability. More specifically, the machine learning model generation unit 180 generates the machine learning model trained on the relationship between the health data in the age group of the transition source and the health data in the age group after transition based on the state transition probability. The machine learning model generation unit 180 is an example of a machine learning model generation means.

### (Prediction Phase)

The acquisition unit 110 acquires target data that is health data of a target person.

The prediction unit 140 estimates the future health state of the target person using the machine learning model. Specifically, the prediction unit 140 predicts the transition of the target data. For example, the prediction unit 140 inputs the target data and the age of the target person to the machine learning model. The health data in the age group equal to or older than the age of the target person is output by the machine learning model. That is, the health data in a case where the target person reaches the age after the lapse of a predetermined period of time or more is output. The prediction unit 140 outputs the health data as future health data of the target person.

For example, it is assumed that the target person is 51 years old. It is assumed that health data in a case where the target person is 70 years old is predicted. In this case, the prediction unit 140 inputs, for example, information indicating that the age is 51 and the target data to the machine learning model. At this time, the machine learning model outputs the health data regarding a 70-year-old person, which is the age group as the transition destination of the input health data. The prediction unit 140 outputs the output health data as health data in a case where the target person is 70 years old.

The machine learning model may be a model that outputs health data after the lapse of a specific period of time with respect to the target data. For example, the machine learning model may output health data in a case where the target person reaches an age group after 20 years. The machine learning model may be a model that outputs a transition of health data until a specific period of time has elapsed. For example, the prediction unit 140 may output the transition of the health data until the target person reaches the age group after 20 years.

The prediction unit 140 predicts data in the prediction-target distribution, which is the transition destination of the data corresponding to the health data of the target person, using the machine learning model.

### [Operation Example of Prediction Device 101]

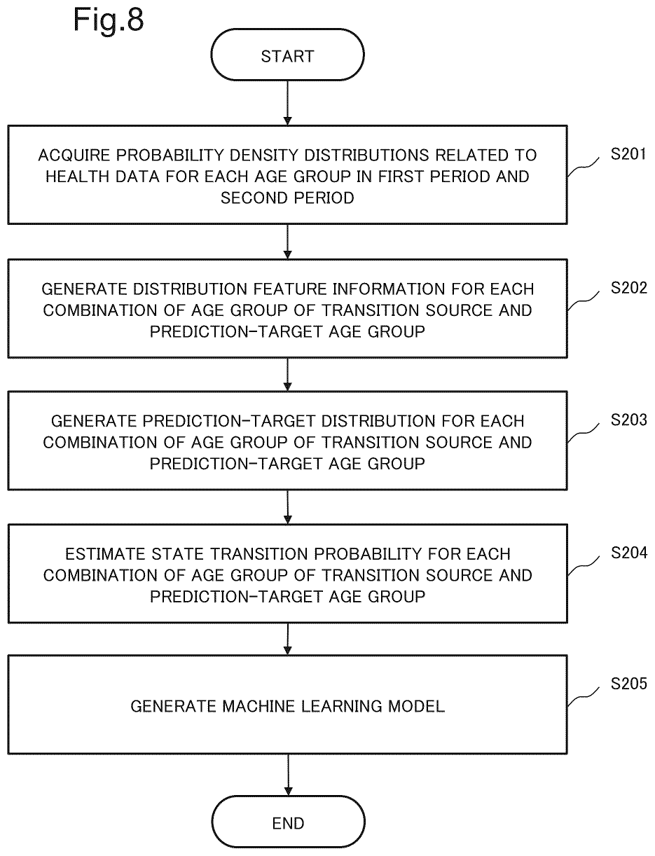

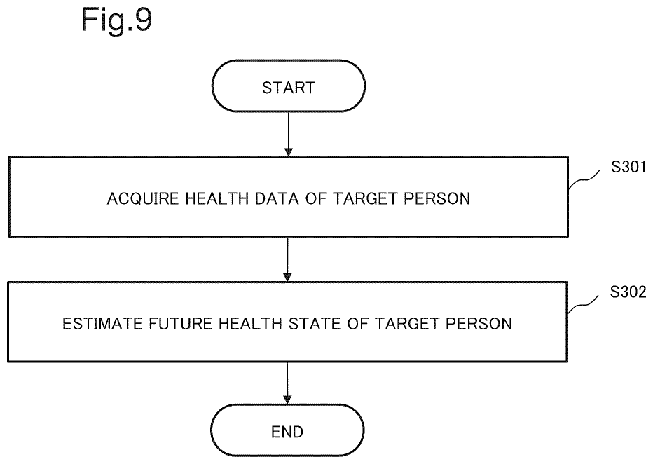

Next, an example of an operation of the prediction device 101 will be described with reference to FIGS. 8 and 9.

FIG. 8 is a third flowchart illustrating an example of the operation of the prediction device 101. Specifically, FIG. 8 is a flowchart illustrating an example of the operation of the prediction device 101 in the generation phase. In the operation example of FIG. 8, it is assumed that a probability density distribution related to health data for each age group is stored in the storage device 190 in advance.

The acquisition unit 110 acquires probability density distributions related to the health data for each age group in the first period and the second period (S201). For example, the acquisition unit 110 acquires the probability density distributions related to the health data for each one-year age in the first period and the second period.

The calculation unit 120 generates the distribution feature information for each combination of the age group of the transition source and the prediction-target age group (S202). For example, it is assumed that a distribution of health data for each one-year age from 20 to 80 is acquired. In a case where age 20 is set as the age group of the transition source, the calculation unit 120 calculates the distribution feature information when each age group from 21 to 80 is set as the prediction-target age group. The calculation unit 120 similarly calculates the distribution feature information also in a case where the age group of the transition source is changed from 21 to 79.

The generation unit 130 generates the prediction-target distribution for each combination of the age group of the transition source and the prediction-target age group (S203).

The estimation unit 160 estimates the state transition probability for each combination of the age group of the transition source and the prediction-target age group (S204). Specifically, the estimation unit 160 uses the prediction-target distribution for each combination of the age group of the transition source and the prediction-target age group to estimate the state transition probability for each combination of the distribution of the transition source and the prediction-target distribution.

The machine learning model generation unit 180 generates a machine learning model based on the estimated state transition probability (S205). Specifically, the machine learning model generation unit 180 generates the machine learning model trained on the relationship between the health data in the age group of the transition source and the health data in the age group after transition based on the state transition probability.

FIG. 9 is a fourth flowchart illustrating an example of the operation of the prediction device 101. Specifically, FIG. 9 is a flowchart illustrating an example of the operation of the prediction device 101 in the prediction phase.

The acquisition unit 110 acquires target data that is health data of a target person (S301). For example, the acquisition unit 110 acquires the target data from the terminal device.

The prediction unit 140 predicts the future health state of the target person using the machine learning model (S302). Specifically, the prediction unit 140 inputs information indicating the age of the target person and the target data to the machine learning model. The health data is output by the machine learning model. The prediction unit 140 outputs the output health data as health data in a case where the target person reaches an age after a predetermined period of time or more has elapsed.

The present operation example is merely an example. That is, the operation of the prediction device 101 of the present disclosure is not limited to this example.

The prediction device 101 estimates the state transition probability based on the transition of the distribution of the health data for each combination of the age group of the transition source and the prediction-target age group in the health data in the first period. The prediction device 101 generates a machine learning model trained on the relationship between the health data in the age group of the transition source and the health data in the age group after transition based on the state transition probability. The prediction device 101 predicts data in the prediction-target distribution, which is the transition destination of the data corresponding to the health data of the target person, using the machine learning model.

### First Modification Example

In the present disclosure, the example in which the prediction device estimates the data transition based on the health data has been mainly described. That is, the example in which the prediction device is used in the healthcare or medical field has been mainly described. However, the example to which the prediction device is applied is not limited thereto. For example, the prediction device may also be applied to a case of estimating state transitions of various machines.

For example, in a case where the measurement data measured for the operating state of the machine is acquired, the prediction device may receive, as the dataset for each stage, the measurement data of each state based on the secular change from the state in which the machine normally operates to the state in which the machine fails. The prediction device may estimate the state transition probability based on the distribution of the measurement data between the states. The prediction device may output the state transition probability related to the transition between the states.

In JP 2018-194904 A, the input vector is generated from, for example, receipt data, medical examination data, or questionnaire data at a plurality of past time points regarding the same subject. Training is performed using the input vectors.

That is, in JP 2018-194904 A, temporal data obtained by observing the same person for a certain period of time is used to predict the state of the subject. In a case where long-term prediction such as prediction 10 years in the future or prediction 20 years in the future is performed, temporal data regarding the same person for a long period of time is required to perform a similar prediction method. In this case, it is necessary to perform long-term observation on the same person. Therefore, there is a high possibility that it is difficult to acquire such temporal data regarding the same person for a long period of time.

One example of an object of the present disclosure is to address the above-described problems and provide a prediction device and the like capable of predicting a health state after a predetermined period of time even in a case where there is no temporal data for a predetermined period of time.

According to the present disclosure, even in a case where there is no temporal data regarding the same person, it is possible to estimate a state transition in consideration of a past state.

### <Hardware Configuration Example of Prediction Device>

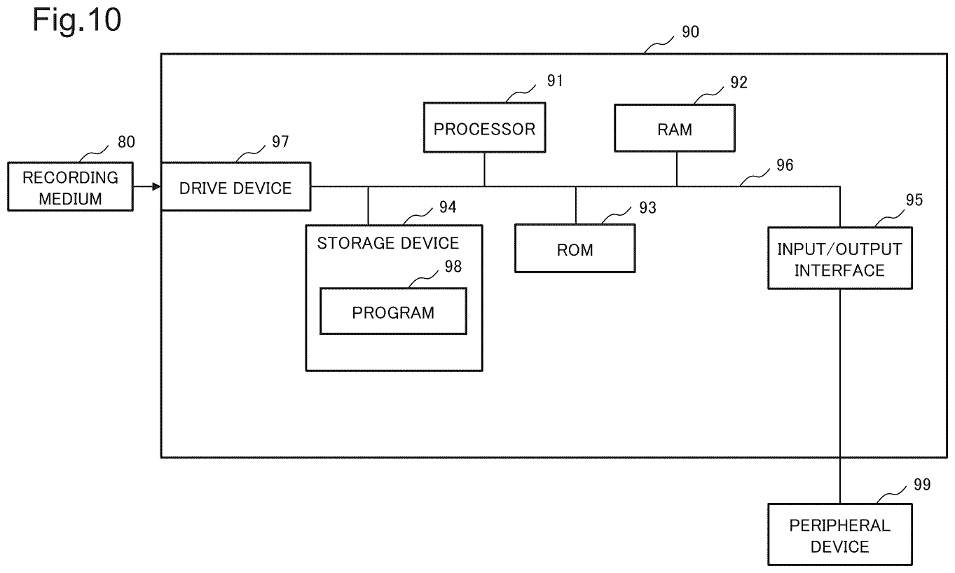

Hardware constituting the prediction devices of the first, second, and third example embodiments will be described. FIG. 10 is a block diagram illustrating an example of a hardware configuration of a computer device constituting the prediction device according to each example embodiment. In a computer device 90, the prediction device and the prediction method described in each example embodiment and each modification example are implemented. For example, the prediction device and the like described in each example embodiment and each modification example may have the hardware configuration illustrated in FIG. 10.

As illustrated in FIG. 10, the computer device 90 includes a processor 91, a random access memory (RAM) 92, a read only memory (ROM) 93, a storage device 94, an input/output interface 95, a bus 96, and a drive device 97. The prediction device and the like may be implemented by a plurality of electric circuits.

The storage device 94 stores a program (computer program) 98. The processor 91 executes the program 98 of the present prediction device using the RAM 92. Specifically, for example, the program 98 includes a program that causes a computer to execute the processing illustrated in FIGS. 2, 6, and 8. When the processor 91 executes the program 98, the function of each configuration of the present prediction device is implemented. The program 98 may be stored in the ROM 93. The program 98 may be recorded in a recording medium 80 and read by the drive device 97, or may be transmitted from an external device (not illustrated) to the computer device 90 via a network (not illustrated).

The input/output interface 95 exchanges data with a peripheral device (such as a keyboard, a mouse, or a display device) 99. The input/output interface 95 functions as a means for acquiring or outputting data. The bus 96 connects the components with each other.

There are various modification examples of the method for implementing the prediction device. For example, each of the components included in the prediction device can be implemented as a dedicated device. The prediction device can be implemented based on a combination of a plurality of devices.

A processing method for causing a recording medium to record a program for implementing each component in the function of each example embodiment, reading the program recorded in the recording medium as a code, and causing a computer to execute the program is also included in the scope of each example embodiment. That is, a computer-readable recording medium is included in the scope of each example embodiment. The recording medium recording the above-described program and the program itself are also included in each example embodiment.

The recording medium is, for example, a floppy (registered trademark) disk, a hard disk, an optical disk, a magneto-optical disk, a compact disc (CD)-ROM, a magnetic tape, a nonvolatile memory card, or a ROM, but is not limited to this example. The program recorded in the recording medium is not limited to a program for executing processing by itself, and programs that run on an operating system (OS) to execute processing in cooperation with other software and functions of an extension board are also included in the scope of each example embodiment.

While the present invention has been particularly shown and described with reference to example embodiments thereof, the present invention is not limited to these example embodiments. It will be understood by those of ordinary skill in the art that various changes in form and details may be made therein without departing from the spirit and scope of the present invention as defined by the claims.

The above-described example embodiments and modification examples can be appropriately combined with each other.

Some or all of the above-described example embodiments may be described as the following supplementary notes, but are not limited to the following.

### SUPPLEMENTARY NOTES

### Supplementary Note 1

A prediction device including:
an acquisition unit that acquires health data being data regarding health of a plurality of persons in a first period and a second period, the second period being a period that is a predetermined duration before the first period;
a calculation unit that calculates distribution feature information indicating a feature related to a distribution of health data of a first age group in the first period based on a relationship between the distribution of the health data of the first age group corresponding to an age of a target person, which is the distribution of the health data in the first period, and the distribution of the health data of the first age group or the distribution of the health data of an age group corresponding to the predetermined duration before the first age group, which is the distribution of the health data in the second period;
a generation unit that generates a prediction-target distribution by integrating a distribution of health data of a prediction-target age group being the distribution in the first period and the distribution feature information, the prediction-target age group corresponding to the age of the target person in a case where the predetermined duration or more has elapsed; and
a prediction unit that predicts the health data in the prediction-target distribution, the health data in the prediction-target distribution being a transition destination of data corresponding to the health data of the target person, in the distribution of the health data of the first age group in the first period.

### Supplementary Note 2

The prediction device according to Supplementary Note 1, further including
a state transition model generation unit that generates a state transition model based on a relationship between a distribution of health data of one age group in the second period and a distribution of health data of another age group in the first period, the state transition model being a model for predicting a transition of a distribution in a case of a transition from the one age group to the another age group, the another age group being an age group after the predetermined duration of the one age group; and
wherein the calculation unit calculates, as the distribution feature information, a post-transition distribution indicating a transition destination of the distribution of the health data of the first age group in the first period in a case of the transition from the first age group to the prediction-target age group, using the state transition model.

### Supplementary Note 3

The prediction device according to Supplementary Note 1,
in which the calculation unit calculates, as the distribution feature information, a first difference being a difference between the distribution of the health data of the first age group in the first period and the distribution of the health data of the first age group in the second period.

### Supplementary Note 4

The prediction device according to Supplementary Note 3,
wherein the calculation unit calculates the distribution feature information by combining the first difference and the second difference, and
the second difference is a difference between the distribution of the first age group in the second period and the distribution of the prediction-target age group in the second period.

### Supplementary Note 5

The prediction device according to Supplementary Note 1,
in which the calculation unit calculates, as the distribution feature information, a difference between a prediction distribution of the first age group predicted based on a distribution of an age group the predetermined duration before the first age group in the second period and the distribution of the first age group in the first period.

### Supplementary Note 6

The prediction device according to Supplementary Note 1, further including
a classification unit that classifies data in each distribution of health data for each age group in each period into data groups,
wherein the prediction unit predicts a data group of health data in the prediction-target distribution, the data group of health data in the prediction-target distribution being a data group as the transition destination of the data group obtained by classifying the health data of the target person, in the distribution of the health data of the first age group in the first period.

### Supplementary Note 7

The prediction device according to Supplementary Note 1, further including
an estimation unit that estimates a state transition probability based on a transition from the distribution of the first age group in the first period to the prediction-target distribution,
in which the estimation unit estimates the state transition probability using an optimal transport algorithm that calculates a set of pre-transport data and destination data, which optimizes a transport cost from the distribution of the first age group to the prediction-target distribution.

### Supplementary Note 8

The prediction device according to Supplementary Note 7, further including
a machine learning model generation unit that generates a machine learning model,
wherein the estimation unit estimates the state transition probability based on a transition of the distribution of the health data for each combination of an age group of a transition source and the prediction-target age group in the health data in the first period,
the machine learning model generation unit generates a machine learning model trained on a relationship between the health data in the age group of the transition source and the health data in the age group after transition based on the state transition probability, and
the prediction unit predicts data in the prediction-target distribution, which is the transition destination of the data corresponding to the health data of the target person, using the machine learning model.

### Supplementary Note 9

A prediction method including:
acquiring health data being data regarding health of a plurality of persons in a first period and a second period, the second period being a period that is a predetermined duration before the first period;
calculating distribution feature information indicating a feature related to a distribution of health data of a first age group in the first period based on a relationship between the distribution of the health data of the first age group corresponding to an age of a target person, which is the distribution of the health data in the first period, and the distribution of the health data of the first age group or the distribution of the health data of an age group corresponding to the predetermined duration before the first age group, which is the distribution of the health data in the second period;
generating a prediction-target distribution by integrating a distribution of health data of a prediction-target age group being the distribution in the first period and the distribution feature information, the prediction-target age group corresponding to the age of the target person in a case where the predetermined duration or more has elapsed; and
predicting the health data in the prediction-target distribution, the health data in the prediction-target distribution being a transition destination of data corresponding to the health data of the target person, in the distribution of the health data of the first age group in the first period.

### Supplementary Note 10

A non-transitory computer-readable recording medium storing a program that causes a computer to execute:
acquiring health data being data regarding health of a plurality of persons in a first period and a second period, the second period being a period that is a predetermined duration before the first period;
calculating distribution feature information indicating a feature related to a distribution of health data of a first age group in the first period based on a relationship between the distribution of the health data of the first age group corresponding to an age of a target person, which is the distribution of the health data in the first period, and the distribution of the health data of the first age group or the distribution of the health data of an age group corresponding to the predetermined duration before the first age group, which is the distribution of the health data in the second period;
generating a prediction-target distribution by integrating a distribution of health data of a prediction-target age group being the distribution in the first period and the distribution feature information, the prediction-target age group corresponding to the age of the target person in a case where the predetermined duration or more has elapsed; and
predicting the health data in the prediction-target distribution, the health data in the prediction-target distribution being a transition destination of data corresponding to the health data of the target person, in the distribution of the health data of the first age group in the first period.

Some or all of the configurations described in Supplementary Notes 2 to 8 dependent on the above-described Supplementary Note 1 can also be dependent on Supplementary Notes 9 and 10 by the same dependency relationship as in Supplementary Notes 2 to 8. Some or all of the configurations described as a Supplementary Note can be similarly dependent on various recording means or systems for recording various hardware, software, and software without departing from the above-described example embodiments.

1. A prediction device comprising:
a memory; and
at least one processor coupled to the memory;
the at least one processor performing operations to:
acquire health data being data regarding health of a plurality of persons in a first period and a second period, the second period being a period that is a predetermined duration before the first period;
calculate distribution feature information indicating a feature related to a distribution of health data of a first age group in the first period based on a relationship between the distribution of the health data of the first age group corresponding to an age of a target person, which is the distribution of the health data in the first period, and the distribution of the health data of the first age group or the distribution of the health data of an age group corresponding to the predetermined duration before the first age group, which is the distribution of the health data in the second period;
generate a prediction-target distribution by integrating a distribution of health data of a prediction-target age group being the distribution in the first period and the distribution feature information, the prediction-target age group corresponding to the age of the target person in a case where the predetermined duration or more has elapsed; and
predict the health data in the prediction-target distribution, the health data in the prediction-target distribution being a transition destination of data corresponding to the health data of the target person, in the distribution of the health data of the first age group in the first period.

2. The prediction device according to claim 1, wherein the at least one processor further performs operation to:
generate a state transition model based on a relationship between a distribution of health data of one age group in the second period and a distribution of health data of another age group in the first period, the state transition model being a model for predicting a transition of a distribution in a case of a transition from the one age group to the another age group, the another age group being an age group after the predetermined duration of the one age group; and
calculate, as the distribution feature information, a post-transition distribution indicating a transition destination of the distribution of the health data of the first age group in the first period in a case of the transition from the first age group to the prediction-target age group, using the state transition model.

3. The prediction device according to claim 1, wherein the at least one processor further performs operation to:
calculate, as the distribution feature information, a first difference being a difference between the distribution of the health data of the first age group in the first period and the distribution of the health data of the first age group in the second period.

4. The prediction device according to claim 3, wherein the at least one processor further performs operation to:
calculate the distribution feature information by combining the first difference and the second difference, wherein
the second difference is a difference between the distribution of the first age group in the second period and the distribution of the prediction-target age group in the second period.

5. The prediction device according to claim 1, wherein the at least one processor further performs operation to:
calculate, as the distribution feature information, a difference between a prediction distribution of the first age group predicted based on a distribution of an age group the predetermined duration before the first age group in the second period and the distribution of the first age group in the first period.

6. The prediction device according to claim 1, wherein the at least one processor further performs operation to:
classify data in each distribution of health data for each age group in each period into data groups; and
predict a data group of health data in the prediction-target distribution, the data group of health data in the prediction-target distribution being a data group as the transition destination of the data group obtained by classifying the health data of the target person, in the distribution of the health data of the first age group in the first period.

7. The prediction device according to claim 1, wherein the at least one processor further performs operation to:
estimate a state transition probability based on a transition from the distribution of the first age group in the first period to the prediction-target distribution,
wherein the state transition probability is estimated by using an optimal transport algorithm calculating a set of pre-transport data and destination data, which optimizes a transport cost from the distribution of the first age group to the prediction-target distribution.

8. The prediction device according to claim 7, wherein the at least one processor further performs operation to:
estimate the state transition probability based on a transition of the distribution of the health data for each combination of an age group of a transition source and the prediction-target age group in the health data in the first period,
generate a machine learning model trained on a relationship between the health data in the age group of the transition source and the health data in the age group after transition based on the state transition probability, and
predict data in the prediction-target distribution, which is the transition destination of the data corresponding to the health data of the target person, using the machine learning model.

9. A prediction method comprising:
acquiring health data being data regarding health of a plurality of persons in a first period and a second period, the second period being a period that is a predetermined duration before the first period;
calculating distribution feature information indicating a feature related to a distribution of health data of a first age group in the first period based on a relationship between the distribution of the health data of the first age group corresponding to an age of a target person, which is the distribution of the health data in the first period, and the distribution of the health data of the first age group or the distribution of the health data of an age group corresponding to the predetermined duration before the first age group, which is the distribution of the health data in the second period;
generating a prediction-target distribution by integrating a distribution of health data of a prediction-target age group being the distribution in the first period and the distribution feature information, the prediction-target age group corresponding to the age of the target person in a case where the predetermined duration or more has elapsed; and
predicting the health data in the prediction-target distribution, the health data in the prediction-target distribution being a transition destination of data corresponding to the health data of the target person, in the distribution of the health data of the first age group in the first period.

10. A non-transitory computer-readable recording medium storing a program that causes a computer to execute:
acquiring health data being data regarding health of a plurality of persons in a first period and a second period, the second period being a period that is a predetermined duration before the first period;
calculating distribution feature information indicating a feature related to a distribution of health data of a first age group in the first period based on a relationship between the distribution of the health data of the first age group corresponding to an age of a target person, which is the distribution of the health data in the first period, and the distribution of the health data of the first age group or the distribution of the health data of an age group corresponding to the predetermined duration before the first age group, which is the distribution of the health data in the second period;
generating a prediction-target distribution by integrating a distribution of health data of a prediction-target age group being the distribution in the first period and the distribution feature information, the prediction-target age group corresponding to the age of the target person in a case where the predetermined duration or more has elapsed; and
predicting the health data in the prediction-target distribution, the health data in the prediction-target distribution being a transition destination of data corresponding to the health data of the target person, in the distribution of the health data of the first age group in the first period.
